(19) United States
(12) Plant Patent Application Publication
(10) Pub. No.: US 2006/0031972 P1
(43) Pub. Date: Feb. 9, 2006

(54) WALNUT TREE NAMED 'GILLET'

(50) JUGLANS REGIA; WALNUT

(75) Inventors: Gale McGranahan, Davis, CA (US); Charles Leslie, Davis, CA (US)

Correspondence Address: MORRISON & FOERSTER LLP, 425 MARKET STREET, SAN FRANCISCO, CA, 94105-2482, US

(73) Assignee: UNIVERSITY OF CALIFORNIA, Oakland, CA (US)

(21) Appl. No.: 10/944,025

(22) Filed: Sep. 17, 2004

Related U.S. Application Data
(63) Continuation-in-part of application No. 10/912,781, filed on Aug. 6, 2004, now abandoned.

(51) Int. Cl. A01H 5/00

(52) U.S. Cl. PLT/154

(57) ABSTRACT

A new and distinct variety of walnut tree denominated ‘Gillet’ is described. This new cultivar comes into bearin young, produces well mid-season, and bears a jumbo sized nut with lid colored kernels of uniform size. The new ciltivar can be harvested prior to ‘Chandler’ and firthermore shows low susceptibility to walnut blight

### CROSS-REFERENCE TO RELATED APPLICATION

The present application is a continuation-in-part of U.S. patent application Ser. No. 10/912,781, titled WALNUT TREE NAMED ‘GILLET’, filed on Aug. 6, 2004, which is hereby incorporated by reference in its entirely.

### FIELD OF THE INVENTION

Botanical/commercial classification: (Juglans regia)/new English walnut variety, Varietal denomination: cv. (Qillet.

### BACKGROUND OF THE INVENTION

The present invention relates to a new and d ot variety of walnut tree Juglans regia which has been denominated varietally as ‘Gillet,’ and more particularly to such a walnut tree which has a harvest date approximately two weeks earlier than the walnut bee variety ‘Chandler’ (U.S. Plant Pat. No. 4,388) and which further produces a walnut th is jumbo in m with light colored kernels and which can be processed in shell or cracked.

It has long been recognized as desirable to provide walnut trees bearing large crops which are ripe for commercial harvesting and shipment midseason and exhibit low susceptibility to walnut blight The tree oft. proseat variety, ‘Gillet,’ produces a nut which is similar in some respects to common walnut tree varieties such as ‘Chandler,’ (U.S. Plant Pat. No. 4,388). However the new variety is ready for harvest approximately two weeks before ‘Chandler,’ and ten days after the common rererence cultivar ‘Payne’ (not pataed).

The new Juglans regia wanut tree of the present invention was created at Davis, Calif. in 1995 by a controlled cross of the cuitivar ‘Chico’ and UC76-80 (neither patented). The pedigree is illustrated (FIG. 1).

Seeds from the cross were planted and the resulting 37 trees were carefully observed along with other trees in the walnut breeding program. When they began to bear nuts, data were collected annually on leafing date, first peak and last female flower bloom, first peak and last male bloom, blight severity and yield (Table 1). Nuts were sampled, cracked, and data was collected on shell appearance, shell thickness, shell integrity, shell strength, nut weight, kernel weight, pcent kernel, ease of kernel removal, kernel color, and percent kernel shrivel (Table 2). A single tree was s*lecte& from among progeny of this controlled cross based on its superior attributes. fhis selection was org y designated ‘UC95-22-26,’ and is now designed the ‘Gillet’ cultivar after Felix Gillet, a historical figure said to be responsible for introducing varieties suitable for the northeem half of California and therefore responsible for the growth of the walnut industry early in the 20^{th} century. Compared to ‘Gillet’ the parent UC76-80 is protandrous and the nut has a weaker shell; the parent ‘Chico’ has smaller nuts than ‘Gillet’ with a more difficult to extract kernel.

The new cultivar of the present invention has been propagated by g at Davis, Calif. on ‘Paradox’ hybrid rootstock The distinctive caracteristies of the new cultivar have been found to be stable and are transmitted to the new trees when asexually propagated.

### SUMMARY OF THE INVENION

It was found that the new Juglans regia cultivar of the present invention exhibits the following combination of characteristics:

a) Comes into bearing young, with an excellent yield at age 3 years;

b) forms jumbo-sized walnuts the possess light-colored kernels with little size variation in a given harvest;

c) can be processed inshell or cracked;

d) bears fruit laterally;

e) yields a walnut crop that can be harvested 2 weeks prior to ‘Chandler’ (U.S. Plant Pat. No. 4,388).

f) is protogynous, bearing female flowers before male flowers; and

g) exhibits low susceptibility to blight.

### BRIEF DESCRIPJMON OF TIBE TABLES

Table 1 shows comparative tree evaluatins.

Table 2 shows nut and kernel fraits.

### BRIEF DESCRIPTION OF THIE DRAWINGS

FIG. 1—sows the pedigree of the ‘Gillet’ walnut.

FIG, 2—shows a tree of the ‘Gillet’ walnut at seven years.

FIG. 3—shows a near view of the typical current season's stem of the ‘Cilet’ walnut.

FIG. 4—shows a near view ofthe leaves of the ‘Gillet’ walnut.

FIG. 5—shows a near view oftthe nuts in the hull of the ‘Gillet’ walnut just prior to maturity.

FIG. 6—shows nuts in the hull of the ‘Gillet’ walnut at maturity.

FIG. 7—shows kernel and nut of the ‘Gillet’ walut.

### BOTANICAL DESCRIPTION OF THE PLANT

The description is based ona uafed walnut on its own roots and trees propagated by grafting on Paradox rootstock and growing in an orchard at Davis, Calif. Data were collected on the own rooted tree fro 1999, at age 3 years, to 2003, age seven. In 2002, scionwood from this tree was collected and grufted onto Paradox rootstock for further evaluation in three sites: Davis, Chico and Kearney.

The Munsell Book of Color is used in the ideffifcation of color, Also; common color terms are to be accorded their ordinary dictionary significance.
Botanical classification: Juglans regia.
Female parent: ‘Chico’.
Male parent: UC76-80.

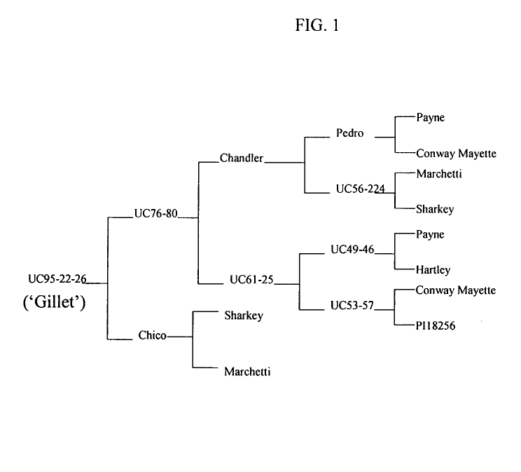

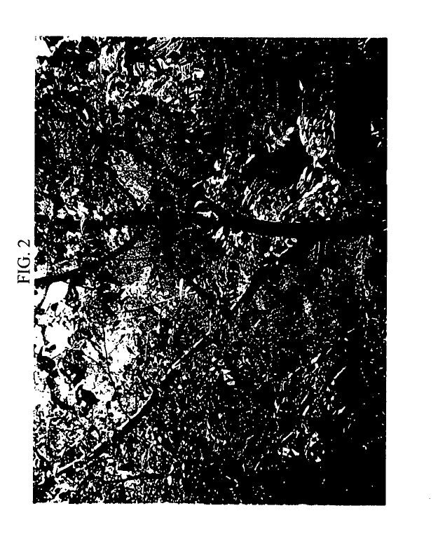

The pedigree is shown (FIG. 1).

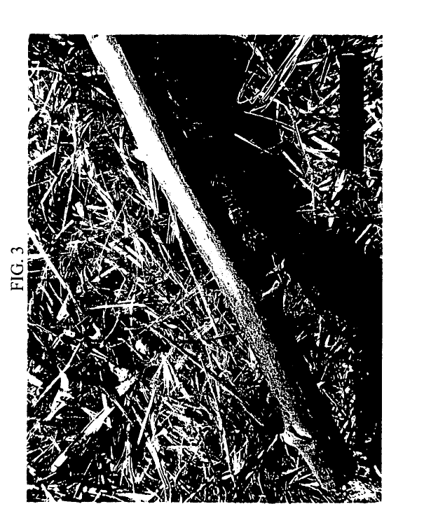

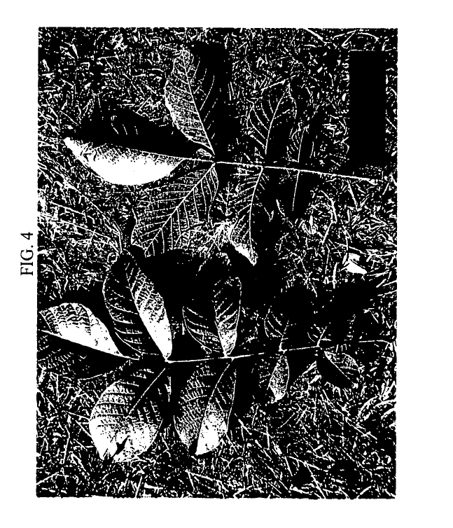

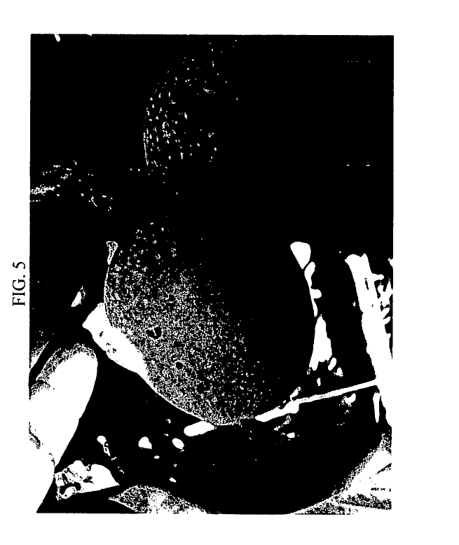

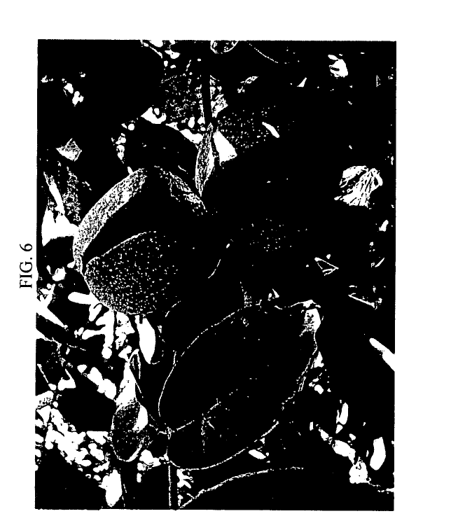

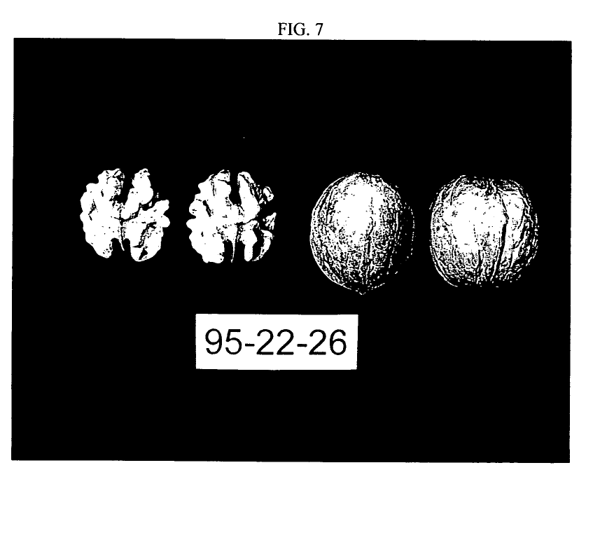

Plant: The growth habit of the tree is illustrated. in FIG. 2. This 7 year old tree was approximately 22 feet in height with a canopy diameter of approxiately 19 feet. The trnk diameter at 30 cm above t ground is approximately 20 cm. The silvery grey bark is typical of Juglans regia. The young bark is brown (2.9GY 2.3/3.6) with raised white lenticels (FIG. 3) and the older bark is grey (5Y 7.5/2) with lighter striations (7 YR 8/2). On one year-old bark there are about 12 lenticels per 2.5 cm of stem measnring approximately 1.5 cm in diameter. ‘Gillet’s lenticels are oval, measure 2-10 mm by 1-2 mm and are light grey yellow brown (9.7YR 4.6/2.1). ‘Cillet’ has vigor similar to the ‘Serr’ variety. The surie texture of trunk branch, leaflets, uill and kernel are smooth.
Foliage: The dark green foliage is illustrated (FIG. 4) and is typical of Juglans regia. Leaf out during 1994-2003 has occurred on March 30 on the average. For comparative purposes the ‘Payne’ cultivar leafed out 9 days earlier and the ‘Chandler’ cultivar leafed out 7 days later udnrithe same years. The typical leaf coloration is green, 6.1GY 3.2/5.8, on the upper surface, and slightly lighter (5.0GY 4.5/8.2) on the lower surfhce. The leaves are pinnatey compound with 5-7 (9) leaflets. The full leaf length is approximately 37 cm and the width 26.6 cm. Leaflets are broadly elliptical and entire. The terminal leaflet averages 15 cm in length and 10 cm in vadth. The middle leaflets average 13 cm in length and 7.6 cm in width and the proximal leaflets avierage 8.9 cm in length and 5.4 cm in width. If 7 leaflets are present the fir (proximal)set is smallest averaging 6.3 cm in length and 4 cm in width. The rachis averages 22.4 cm in length and is 1-2 mm in diameter. Petioles average length is 7 cm, 2-4 mm in diameter and 5GY 7/8 in color.
Inflorescence: The tree is relatively precocious, an excellent yield being noted at age 3 years. Male flowers (catkins) were not present until age 5. This delay in male maturity is typical of Juglans regia. The catiin's diameter is about 15 mm and yellow-green (5GY 6/8). Catin length ranges between 7 and 13 cm. From 1999 to 2003, first female bloom occurred on an average on April 4, peak bloom on April 8 and last bloom on April 15. From 2001 to 2003, average male flowering (pollen shedding) began April 10, peaked on April 18 and termated April 27. In this protogynous tree, pollen shedding does not completely cover pistillate bloom suggesng that a pollenizer would be needed for maximum yield in isolated areas. Both ‘UC90-31-10’ (patent application Ser. No. 10/912,852) and ‘Serr’ (unpatented) would be satisfactory pollenizers. The female flowers are typical of Juglans regia with two flowers per inflorcccence borne at both terminal and lateral positions on current season's growth. Approximately 98% of the lateral buds contain inflorescences making yields much greater than trees that only bear flowers terminally. A typical female flower is approximately 5 to 7 mm at anthesis and the floral organs are typical of J. regia. The flowers appear vaseshaped when the two plumose stic arms are curved outwardly. There are no petals. The flowers measure 5-7 nm in length and 3-5 mm in diameter and are yellow-green (5GY 6/8) in color. They are borne usually in twos on a 1 cm spike. The flower fragrance is typical of J. regia and is not noticeably different tha the foliage fragrance.
Walnuts: The new cultivar commonly harvests at least eleven days before ‘Chandler’ and two weeks after ‘Payne’ but may become earlier as the clone ages. During 2003, nuts of this new eultivar were ready for harvest on October 1. This compares with ‘Payne’ that harvested September 21, and ‘Chandler’ that harvested October 19. The new cutivar has excellent yields of jumbo-sized walnuts. The hull is globose, 5 cm×5 cm, 5.8 mm tick and 2.5GY 8/6 in color The apex is between roned and trucate, the base is round and it is approximately 1.3-1.5 mm thick. The round nutshell is tan, relatively smooth, and measures approximately 38.7 mm in length and 38.5 mm in width. The shell is stong and well sealed and the kernel is easy to remove. The kernel weighs 8.2 g and makes up 51.5% of the total nut weight of 16.0 g. Kernel color is considered excellent and scores mostly in tie light to extra light categories of the USDA Standards for Grades of Shelled Walnuts as determined by using the standard Walnut Color Chart for kernels published by the Died Fruit Association of California. In addition kernels of ‘Millet’ scored 55 on the Relative Light Index used by Diamond Walnut of Stockton, Calif.. These values are based on 5 year averages often walnut samples obtained each year from a young tree. Typical kemel demonsions are approximately 31.8 mm in length and 32.3 mm in width. The kernel is essentially round and splits into halves easily, It is plump in comparison to ‘Chandler’. It is typical of commercial in terns of flavor and firnness, the latter varying aecording to the percent moisture after drying.
Hardiness: The non-bearing tree withstood a temperature of 21° F. in 1998.
Chilling requirement: Trees have not shown staggered leafing and bloom, symptoms of lack of chilling who exposed to over 767 cilling hours (hours under 45° F.).
Disease resistance and susceptibility: Susceptibility to walnut blight has been low even though adjacent trees have been severely afflicted. No other unusual resistance or susceptibility to insects and diseases has been observed to date.
Usage: The new cultivar of the present invention provides a mid-season-harvesting walnut eultivar with light colored kernels that can be used cracked or in shell.

What we claim is:

1. A new variety of walnut tree substantally as shown and described herein.
